(19) United States
(12) Patent Application Publication
(10) Pub. No.: US 2014/0154197 A1
(43) Pub. Date: Jun. 5, 2014

(54) Packaged Antiperspirant Compositions

(71) Applicant: The Procter & Gamble Company, Cincinnati, OH (US)

(72) Inventors: David Frederick SWAILE, Cincinnati, OH (US); Barbara Marie MODAFARI, Mason, OH (US); Elton Luis MENON, Mason, OH (US); Jody Lynn HOYING, Maineville, OH (US); Roland STARK, Warfield (GB); Sheena Marie MAHRLE, Liberty Township, OH (US)

(73) Assignee: The Procter & Gamble Company, Cincinnati, OH (US)

(21) Appl. No.: 14/090,175

(22) Filed: Nov. 26, 2013

Related U.S. Application Data
(60) Provisional application No. 61/729,867, filed on Nov. 26, 2012.

(51) Int. Cl. A45D 40/26; A61Q 15/00; B65B 63/08; A61K 8/92

(52) U.S. Cl. CPC A45D 40/26; A61K 8/92; A61Q 15/00; B65B 63/08
USPC 424/66; 424/68; 401/143; 53/440

(57) ABSTRACT

A packaged antiperspirant product is provided. The packaged antiperspirant product includes an anhydrous antiperspirant composition having from 40% to 60%, by weight of the anhydrous antiperspirant composition, of solids. The solids include an antiperspirant active, one or more waxes, and one or more fillers having a total concentration from about 15% to about 35% by weight of the anhydrous antiperspirant composition. The antiperspirant composition also has from about 35% to about 60%, by weight of the composition, of one or more carriers other than water, wherein the one or more carriers comprises a non-volatile silicone fluid. The package includes a container body having an interior chamber storing the anhydrous antiperspirant composition, a dome closing one end of the container body and having a plurality of apertures extending through the thickness of the dome. An elevator and feed screw are disposed within the container body.

### FIELD OF THE INVENTION

The present application is directed to antiperspirant compositions, packaged antiperspirant compositions and methods relating thereto.

### BACKGROUND OF THE INVENTION

There are many types of topical antiperspirant products that are commercially available or otherwise known. Most of these products are formulated as sprays, roll-on liquids, creams like soft solids, or solid sticks, and comprise an astringent material, e.g. zirconium or aluminum salts, incorporated into a suitable topical carrier. These products are designed to provide effective perspiration and odor control while also being cosmetically acceptable during and after application onto the axillary area or other areas of the skin. Some examples of antiperspirant compositions are described in U.S. Pat. Nos. 4,840,789; 4,853,214; 5,019,375; 5,102,656; 5,178,881; 5,294,447; 5,843,414; 6,616,921; 6,426,062; 6,849,251 and 2003/0185777.

Within this product group, antiperspirant soft solids have become increasingly more popular as an effective alternative to antiperspirant sprays and solid sticks. Perspiration and odor control provided by these products can be excellent. Many of these soft solid products, however, may be cosmetically unacceptable to a large number of antiperspirant users because application of these soft solids can be messy, difficult to spread and wash off, and the applied areas often feel wet or sticky, as opposed to light and dry, for several minutes after application. As such, there is a need for improved antiperspirant compositions and methods.

### SUMMARY OF THE INVENTION

In one aspect, a packaged antiperspirant product is provided. The packaged antiperspirant product includes an anhydrous antiperspirant composition having from 40% to 60%, by weight of the anhydrous antiperspirant composition, of solids. The solids include an antiperspirant active, one or more waxes, and one or more fillers having a total concentration from about 15% to about 35% by weight of the anhydrous antiperspirant composition. The antiperspirant composition also has from about 35% to about 60%, by weight of the composition, of one or more carriers other than water, wherein the one or more carriers comprises a non-volatile silicone fluid. The package includes a container body having an interior chamber with the anhydrous antiperspirant composition stored therein, a dome closing one end of the container body and having a plurality of apertures extending through the thickness of the dome. An elevator is disposed within the container body for pushing the anhydrous antiperspirant composition toward the dome. A feed screw threadably engages the elevator for advancing the elevator toward the dome.

### BRIEF DESCRIPTION OF THE DRAWINGS

FIG. 1 is a graph showing tack value (gF, gram Force) as a function of percent solids (antiperspirant active and filler) in a composition;

FIG. 2 is a log scale graph depicting viscosity (Pa·s, Pascal seconds) as a function of shear rate (1/s) of Inventive Example 3, Dove® Clinical Protection, and Secret® Clinical Strength;

FIG. 3A is a picture of antiperspirant compositions with varying waxes as noted;

FIG. 3B is a picture of antiperspirant compositions with varying wax levels tested for simulated application to wet skin as discussed herein;

FIG. 4 is a graph depicting viscosity (Pa·s) versus shear rate (1/s) of Inventive Example 3; and

FIG. 5 is a graph depicting viscosity (Pa·s) versus shear rate (1/s) of Inventive Example 3 when processed in two different manors.

FIG. 6 is perspective view of package suitable for use with antiperspirant compositions described herein.

FIG. 7 is a top plan view of the package of FIG. 6.

FIG. 8 is a cross-sectional side elevational view of the package of FIG. 6, taken along line 8-8 thereof.

### DETAILED DESCRIPTION OF THE INVENTION

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

In all embodiments, all percentages are by weight of the antiperspirant composition (or formulation), unless specifically stated otherwise. All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. The term “room temperature” refers to 25° C. The term “molecular weight” or “M.Wt.” as used herein refers to the number average molecular weight unless otherwise stated.

“Anhydrous” refers to compositions and/or components which are substantially free of or completely free of, water added as a separate component. The composition may, however, still contain water that comes in with the with raw materials (e.g., antiperspirant active, hydrophilic starches, etc.).

“Antiperspirant gel” means a composition comprising an antiperspirant active and which is in the form of water in oil emulsion, wherein the water phase comprises greater than 20% of the antiperspirant composition.

“Antiperspirant stick” means a solid composition comprising an antiperspirant active and having a rheology sufficient for dispensing from a package that does not have a dome containing a plurality of apertures.

“Emollient” means a material that creates a lubricious feel or softening effect on skin. Emollients may be liquids or semi-solids at room temperature, such as gel.

“High shear rate viscosity” means the viscosity of a composition at a shear rate of approximately 315 1/S (310 to 320 1/S) measured using a rheometer.

“Liquid” means a material that is in a liquid or flowable state at room temperature as a raw material.

“Low shear rate viscosity” means the viscosity of a composition at a shear rate of approximately 0.01 1/S (0.0075 to 0.0125 1/S) measured using a rheometer.

“Non-polar” means materials having a Hildebrand solubility parameter of less than 16 MPa^{1/2}.

“Polar” means materials having a Hildebrand solubility parameter of more than 16 MPa^{1/2}.

“Rheometer” means a cone and plate rotational rheometer or a parallel plate rotational rheometer, such as, for example, model TA AR2000 available from TA Instruments, Inc., USA.

“Solids” means particulate raw materials that never dissolve in the antiperspirant composition during processing and those materials that start as a solid at room temperature then dissolve or melt at high temperature and return to a solid state when the composition is cooled back to room temperature.

“Substantially free of” means 1% or less or about 0.1% or less of a stated ingredient. “Free of” refers to no detectable amount of the stated ingredient or thing.

“Syneresis” means the separation or weeping of oil/liquid from an antiperspirant composition. This may occur, for example, when the antiperspirant composition is extruded thru an apertured dome of a package or during shipment from agitation.

“Viscosity” means dynamic viscosity (measured in centipoise, cPs, or Pascal-second, Pa·s) of a material or composition at approximately 25° C. and ambient conditions.

“Volatile” means a material that has a boiling point less than 250° C. at atmospheric pressure. “Non-volatile” means a material that has a boiling point greater than 250° C. at atmospheric pressure.

“Water insoluble hydrophilic oil” refers to an oil that is a liquid at room temperature, has a Hildebrand solubility parameter of greater than 16 MPa^{1/2} and a water solubility of less than 1% wt in water at 20° C. Solubility parameters for the water insoluble hydrophilic oils and means for determining such parameters are well known in the chemical arts. A description of solubility parameters and means for determining them are described, for example, by C. D. Vaughan, “Solubility Effects in Product, Package, Penetration and Preservation” 103 Cosmetics and Toiletries 47-69, October 1988; and C. D. Vaughan, “Using Solubility Parameters in Cosmetics Formulation”, 36 J. Soc. Cosmetic Chemists 319-333, September/October, 1988, which descriptions are incorporated herein by reference.

“Wax” refers to materials of any molecular weight that are a solid at room temperature, melt at a temperature between about 40° C. and 125° C. and return to a solid at room temperature.

### I. Soft Solid Antiperspirant Compositions

Soft solid, as an antiperspirant form, is well liked by many consumers. In fact, once certain consumers use this form, they are very loyal to it in spite of its drawbacks. At least partially due to formulation limitations, current soft solid products may often leave a tacky feeling after application. In contrast, some consumers desire an antiperspirant composition that approaches feeling more like a powder (light and dry) at application. There may also be some difficulties applying antiperspirant compositions through hair and to wet skin. This can be a particular problem for male users, as they may tend to have more hair in the axillia area and/or may be less prone to dry the axillia prior to applying an antiperspirant composition. The present inventors have discovered at least some of these drawbacks can be overcome by balancing the solids level and type, viscosity, wax type and level, and/or hydrophilic water insoluble oil levels.

While soft solid is generally used to describe any form between a liquid and a solid, it is used herein to describe those compositions which can be effectively dispensed from a package having an elevator and a dome with a plurality of apertures that can be slots, round holes or otherwise, some examples of which are described hereafter (one example would also be the slotted applicator like those currently used on Secret® Clinical Strength products). A soft solid antiperspirant composition can be effectively delivered from this type of package if it has enough structure to prevent the antiperspirant composition from running out of the package during application and not so much structure as to prevent it from being dispensed through the apertures in the dome's surface. Soft solid antiperspirant compositions generally have a continuous thickening structure created by a wax or other thickener that is often created by pouring a molten liquid antiperspirant composition into its package in a molten state at or above its solidification onset temperature so that the final structure of the solid antiperspirant composition is formed without agitation below its temperature of solidification onset. The solidification onset temperature is the temperature at which the composition begins to crystallize and may be measured by the well known technique of Differential Scanning calorimetery (DSC) using, for example, a Perkin-Elmer 7 Series Thermal Analysis System Model DSC7 (at a scan rate of less than 5° C. per minute). Depending on the type of wax, the temperature of solidification onset may be from about 40° C. to about 60° C., and it may be desirable to pour the molten liquid antiperspirant composition into the package at 3° C. to 5° C. or more above the solidification onset temperature (e.g., pouring at temperatures greater than about 40° C. to about 45° C. or from about 40° C. or about 45° C. to about 80° C., preferably from about 40° C. or 45° C. to about 60° C. or about 65° C.). This is in contrast to paste type compositions that are often formed by shearing below the temperature of solidification onset (i.e., as the composition is solidifying) to reduce viscosity by preventing the formation of a continuous thickening structure. In other words, the wax is prevented from forming a strong interconnected crystalline matrix due to shearing during solidification. This may be accomplished by constant stirring of the antiperspirant composition as it cools and solidifies (see, e.g., U.S. Pat. No. 6,849,251, wherein the antiperspirant composition is left to cool to room temperature with constant stirring). The sheared antiperspirant solid can then be scooped, or transferred using a screw extruder in the case of commercial manufacture, into a package. It is believed that such a constant stirring process below the solidification onset temperature may result in a lower viscosity than a non-stirring process below the solidification onset temperature (e.g., compare Examples 3 and 3A). The lower viscosity achieved by such stirring may require high wax concentrations (e.g., greater than 7% w/w) and/or high levels of a hydrophilic oil (e.g., greater than 10% w/w) and/or other thickeners (e.g., silicas) to provide sufficient structure and/or reduce the risk of syneresis. However, higher concentrations of these materials may in turn reduce the efficacy of the antiperspirant composition in some instances compared to an antiperspirant composition having lower concentrations (e.g., compare Examples 4/9 to 10/11). In addition, soft solid antiperspirant compositions, which are in a molten liquid state when poured into the package, easily take on the shape of the interior chamber of the package during the solidification/crystallization process. In contrast, paste type antiperspirant compositions, which may be scooped or extruded into the interior chamber due to their viscosity/more crystallized state, may contain void volumes within the antiperspirant composition, as the composition does not readily take on the form of the interior chamber (absent an additional compressing step to remove the voids, which may lead to syneresis). In some instances, the packaged soft solid antiperspirant compositions described herein may have a total void volume less than 5%, 4%, 3%, 2% or 1% within the total volume of the antiperspirant composition in the package. The total void volume may be determined by means commonly known in the art, such as digital analysis of an x-ray or MRI of the packaged antiperspirant composition.

Soft solid type antiperspirant compositions are also distinguishable from gel type antiperspirant compositions which may have a high internal phase and in some instances are provided in the form of water-in-oil emulsions. As such, the soft solid antiperspirant composition is provided in a form other than a gel.

Soft solid type antiperspirant compositions are preferably anhydrous, although there may be water present that is bound to the antiperspirant active and/or hydrophilic powders (if present). The antiperspirant composition may have a water content of about 10% or less; about 8% or less; about 7% or less; about 5% or less; or about 3% or less from water brought in with the raw materials.

A. Solids

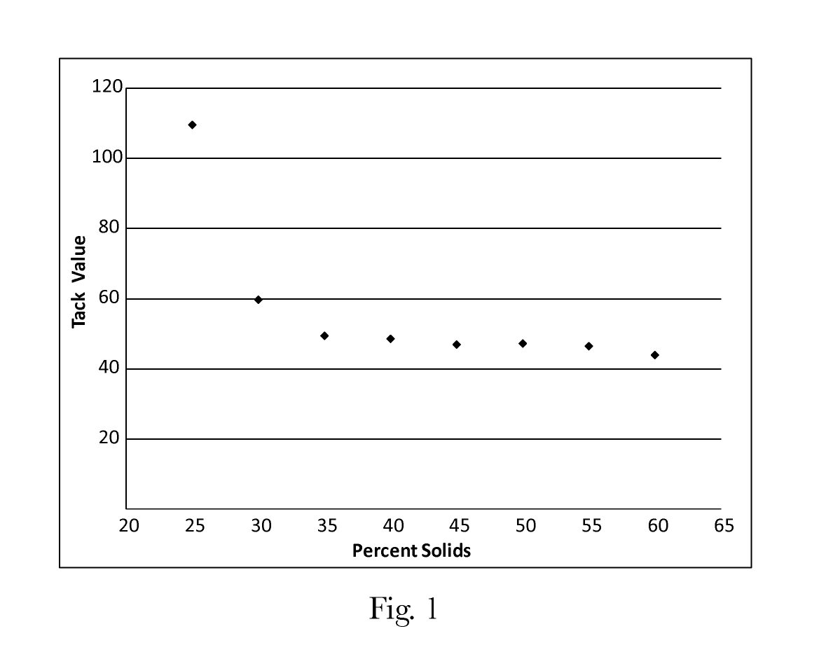

One discovered approach to help alleviate the feeling of tackiness upon application of a soft solid type antiperspirant composition and/or provide a dry/light feel, is to increase the solids level of the antiperspirant composition. It is believed that these solids reduce skin to skin adhesion in the axilla, thereby reducing the feeling of stickiness or tack when the composition is applied. As can be seen from FIG. 1, there is a general reduction in tack in soft solid compositions as the solids level is increased. The antiperspirant compositions of FIG. 1 comprised aluminum chlorohydrate (25% w/w), 50 centistoke dimethicone (5% w/w), stearly alcohol (1% w/w), Syncrowax HGLC (0.5% w/w), hydrogenated high euricic acid rapeseed oil (2% w/w), cyclopentasiloxane (variable w/w %) and hydrophilic tapioca starch (variable w/w %). The compositions representing the data points in FIG. 1 comprised (from left to right in the FIG.): 0% hydrophilic tapioca starch and 66.5% cyclopentasiloxane; 5% hydrophilic tapioca starch and 61.5% cyclopentasiloxane; 10% hydrophilic tapioca starch and 56.5% cyclopentasiloxane; 15% hydrophilic tapioca starch and 51.5% cyclopentasiloxane; 20% hydrophilic tapioca starch and 46.5% cyclopentasiloxane; 25% hydrophilic tapioca starch and 41.5% cyclopentasiloxane; 30% hydrophilic tapioca starch and 36.5% cyclopentasiloxane; and 35% hydrophilic tapioca starch and 31.5% cyclopentasiloxane. The percent solids shown on the x-axis represents the combination of the antiperspirant active and the hydrophilic tapioca starch and does not take into account the wax solids (3.5% w/w/), which were held constant across the compositions. Thus, the solids level was increased by increasing the tapioca starch concentration and reducing the concentration of the cyclopentasiloxane. A similar level of tack is seen between about 40% and 60% solids. Below about 30% solids, the tack increases due to a higher level of liquids in the product. It is believed that liquids are not as efficient at preventing contact between two surfaces in the underarm which can give rise to a higher level of tack. Analysis of two commercial soft solids Secret® Clinical Strength (ingredients are approximately 55% w/w cyclopentasiloxane, 5% w/w dimethicone, 0.5% PPG-14 butyl ether, 3% w/w petrolatum, 4.5% w/w hydrogenated high euricic acid rapeseed oil, 1% w/w Syncrowax HGLC, 3% betacyclodextrin fragrance material, 0.75% perfume and 26% IZAG antiperspirant active, wherein total solids are about 35% w/w and total waxes are about 5.6% w/w) and Dove® Clinical Protection, shows tack values of 111 gF and 116 gF, respectively. Both of these compositions have tack values higher than the soft solid compositions with between about 40% and 60% solids shown in FIG. 1. Thus, antiperspirant compositions may have a tack value of about 110 gF, 100 gF, 80 gF or less, about 70 gF or less, about 60 gF or less, or about 50 gF or less. Other exemplary tack values include from about 10 gF to about 80 gF, from about 10 gF to about 70 gF, from about 10 gF to about 60 gF, from about 40 gF to about 60 gF, or any combination thereof.

The antiperspirant composition may comprise from 40% to about 60%, by weight of the antiperspirant composition, of total solids. Further the antiperspirant composition may comprise from about 45% to about 60% or from about 50% to about 60%, by weight of the composition, of total solids. It is believed that solid concentrations above 60%, even with hydrophobic excipient particles, may become too thick to be effectively rubbed across the entire axillia (e.g., typically 65 cm² to 125 cm²) and/or require undersirably high forces to extrude the antiperspirant composition through the apertured dome of a package. It is believed that solids concentrations less than 40% may tend to provide less of a dry and light feel that more closely approximates application of powder alone. In some instances where it may be desirable to formulate toward the lower end of the solids concentration range described herein, it may also be desirable to then increase the wax concentration toward the higher end of the ranges described herein. Solids may comprise or consist essentially of an antiperspirant active and one or more non-antiperspirant active solids, such as for example one or more waxes and one or more an fillers. Solids may further comprise or consist essentially of fragrance delivery particles.

Fillers

The antiperspirant compositions comprise one or more fillers or excipient powders. The fillers are particulates which would not otherwise substantially thicken an antiperspirant composition at low concentrations (e.g., less than 4% w/w for clays or silicas that: are activated, are in the presence of significant concentrations of free water, milled and/or have a high surface area per gram). Fillers may be chemically inert, reduce the tack of the formula, and/or increase the dry feel of the composition. Fillers exclude clays and silicas added to an antiperspirant composition as bulking or suspending agents, such as organo modified clays activated by a clay activator (e.g, triethyl citrate or methanol or ethanol or propylene carbonate) or silicas with more than 90 to 100 meters² of surface area per gram, such as, for example, fumed silica. Some examples of organo-modified clays include modified bentonite, modified hectorite and modified montorlinite, some examples of which are available under the trade names Bentone 27 (stearalkonium bentonite), Bentone 34 (stearalkonium bentonite), and Bentone 38 (disteardimonium hectorite) from Elmentis Specialities Plc. And Tixogel VPV (quarternium 90-bentonite), Toxogel VZV (stearalkonium benotine), Toxogel LGM (stearalkonium bentonite) and Claytone SO (stearalkonium bentonite) from Southern Clay Products. Non-organo modified clays, such as for example bentonite or hectorite, may be considered fillers and suitable for use herein as the described antiperspirant compositions are anhydrous and therefore may not have enough free water for all of clay to swell to act as a thickener, although it is believed that even such clays may not be preferred in instances where the composition is applied to wet skin and hair unless a polar wax is included in the composition (e.g., for the same reason it is desirable to include a polar wax with a hydrophilic starch). Silicas having less than 90, 80, 70, 60 or 50 meters² of surface area per gram might be used as fillers in some instances. Fillers are also distinct from the antiperspirant active.

Fillers may be hydrophobic or hydrophilic, although hydrophilic powders are generally preferred. Hydrophilic powders may enhance antiperspirant active efficacy by improving water transport into the antiperspirant composition during a sweat event. Hydrophilic powders, at the concentrations described herein, however may also increase the risk of the antiperspirant composition balling up on wet hair/skin surfaces due interaction between the powder, water and the antiperspirant active, as it is believed that men may often apply antiperspirant compositions to wet hair and/or skin surfaces. An antiperspirant composition may comprise one or more fillers which, as a raw material ingredient, comprises or consists essentially of hydrophilic powder, hydrophobic powder, a hydrophobically modified powder, or a mixture thereof. Fillers can have an average particle size of about 50 microns or less and are usually free flowing. The extent of hydrophobicity or hydrophilicity, particle shape and amount of particle to particle interaction are believed to influence the aesthetics and/or efficacy of the soft solid antiperspirant composition.

Fillers or non-antiperspirant active powders can be included at a range of about 15% to about 35% or from about 15% to about 25%, by weight of the antiperspirant composition. The amount of filler can be adjusted based on the weight density of the powder. The filler may be charged or uncharged. In one example, an antiperspirant composition comprises an uncharged hydrophobic filler. The filler may have a desired average particle which can be measured according to methods known in the art, like the laser diffraction method.

It is believed that the selection of the fillers may have an impact on the performance of the antiperspirant composition with respect to wet underarms or wet hair in the underarm. The present inventors discovered that the more hydrophilic the filler, the more likely it is to cause the antiperspirant composition to ball-up when being applied to a wet underarm surface. This may be lessened by including a polar wax (e.g., stearyl alcohol) in the antiperspirant composition (particularly in instances where an unmodified, hydrophilic powder is added as a raw material to antiperspirant composition). The polar wax is believed to coat the hydrophilic filler thereby rendering it moderately hydrophobic in the composition. This modified, moderately hydrophobic powder may sufficiently reduce the propensity for balling during rub in while at the same time is not too hydrophobic that these powders significantly reduce water transport into the composition film. Alternatively, a modified, moderately hydrophobic powder (e.g., Dry Ho TS or Dry Flo PC available for AkzoNobel, which are starches modified with silicone or alkyl groups) may be added as a raw material during the antiperspirant composition making process, in which case a polar wax may be excluded from the antiperspirant composition if desired. So hydrophilic powders rendered moderately hydrophobic during the antiperspirant composition making process and moderately hydrophobic powders added as a raw ingredient tend to work the best where an antiperspirant composition will be applied to wet skin or hair as they tend to give better spreadability on those surfaces, although hydrophilic powders may still be included if desired (particularly for female users who are believed to have less of an issue with wet skin and/hair at time of application). At higher solids concentrations (e.g., 55% to 60% w/w), it may be desirable in some instances for the fillers to consist essentially of or completely of hydrophobic powders (e.g., talc) in order to reduce particle to particle interactions with the antiperspirant active during rub in (and the water bound with the active), or, alternatively, it may be desirable to increase the concentration of a polar wax if a hydrophilic filler is utilized as a raw material.

Some examples of acceptable fillers include tapioca starch, corn starch, oat starch, potato starch, wheat starch, any other starches, cellulose powders, microcrystalline cellulose powders, talc, boron nitriles, polyethylene powders, inorganic powders, perfume delivery vehicles, or combinations thereof. While these fillers may be used, starches and perfume delivery vehicles are more preferred than cellulose powders, which may tend to significantly increase low shear rate viscosity (perhaps due to their rod like shapes, see, e.g., Example 8 which contained 19% cellulose) and/or may hinder rub in/spreadability of the antiperspirant composition at the solids concentrations described herein.

Within the starch family of materials, further useable classes include native or hydrophobically modified starches. Native starches are generally hydrophilic. Hydrophobically modified starches generally have some surface or cross linking treatment that reduces the availability of a fraction of the polar functional groups making them moderately hydrophobic. One filler believed to be particularly suitable for use is a hydrophilic or hydrophobically modified tapioca starch. Thus, in one example, an antiperspirant composition comprises one or more fillers selected from a hydrophilic tapioca starch, a hydrophobically modified tapioca starch, or a combination thereof. Tapioca is a starch which may be extracted from the cassava plant, typically from the root, which may then be processed or modified as known in the art. Tapioca starch particulates may be round to oval in shape and may have an average particle size about 20 microns, which is believed to add in creating a smooth application feel when the product is rubbed on skin. Thus, in one example, an antiperspirant composition comprises a tapioca starch with an average particle size of about 20 microns or less.

A non-limiting example of a hydrophilic tapioca starch material suitable for use is available under the trade name Tapioca Pure available from AkzoNobel. One non-limiting example of a hydrophobically modified tapioca material suitable for use comprises a silicone grafted tapioca starch, which is available under the tradename Dry Flo TS from AkzoNobel of the Netherlands. The INCI name is tapioca starch polymethylsilsesquioxane and may be produced by a reaction of methyl sodium siliconate (polymethylsilsesquioxane) and tapioca starch. This silicone grafted tapioca starch is commercially available as CAS no. 68989-12-8. Other non-limiting examples of hydrophobically modified tapioca starch materials that are suitable for use include Rheoplus PC 541 (Siam Modified Starch), Acistar RT starch (available from Cargill) and Lorenz 325, Lorenz 326, and Lorenz 810 (available from Lorenz of Brazil).

Another filler believed to be suitable for use is a hydrophilic or hydrophobically modified corn starch. Corn starch particulates may roughly approximate a round or oval shape and may have an average particle size about 15 microns. A non-limiting example of a hydrophilic corn starch material is Farmal CS 3757 available from Ingredion, Inc., USA There are a wide variety of modified corn starches that can be used, including but not limited, to Dry Flo PC (aluminum starch succinate) and Dry Flo AF (silicone modified starch) both available from Akzo Nobel.

In some instances, the ratio of filler to antiperspirant active is from about 2:1 to about 1:2.

Waxes

The antiperspirant compositions also comprise one or more waxes. The concentration of the waxes should be high enough to provide sufficient structure (in combination with the high solids level) to the antiperspirant composition while in the package. Also, it may be desirable that the concentrations of waxes are not be too high such that: the low shear rate viscosity of the antiperspirant composition results in difficult dispensing (and possibly then syneresis or separation of the dome from the package), antiperspirant efficacy (as measured by the red dot test) is reduced too much and/or a high tack value (which may detract from a light and dry feel) results. Preferably, the antiperspirant composition has a red dot value greater than 1, 1.5 or 2 and/or a tack value less than about 110 gF, 90 gF or 80 gF, preferably between about 40 gF and about 60 gF.

The wax concentrations may be from about 1%, 1.5% or 2% to about 7%, 6%, 5%, 4% or 3% by weight of the antiperspirant composition. Most preferred are wax concentrations from about 2% to 5% by weight of the composition. It is believed that higher than about 7% by weight of a wax at the solids concentrations described herein, even when incorporating hydrophobic fillers as a raw material, may result in antiperspirant compositions that are too difficult, in some instances, to dispense through an apertured dome. The wax may be polar, non-polar, or a combination thereof. One exemplary wax combination comprises from about 1.5% to about 3%, by weight of the antiperspirant composition, of a non-polar wax, and from about 0.5% to about 2%, by weight of the composition, of a polar wax. More preferably, the wax combination comprises from about 1.5% to about 3%, by weight of the antiperspirant composition, of a non-polar wax, and from about 0.5% to about 2%, by weight of the composition, of a polar wax, particularly for an antiperspirant composition comprising from about 15% to about 25% of a hydrophilic filler added as a raw material.

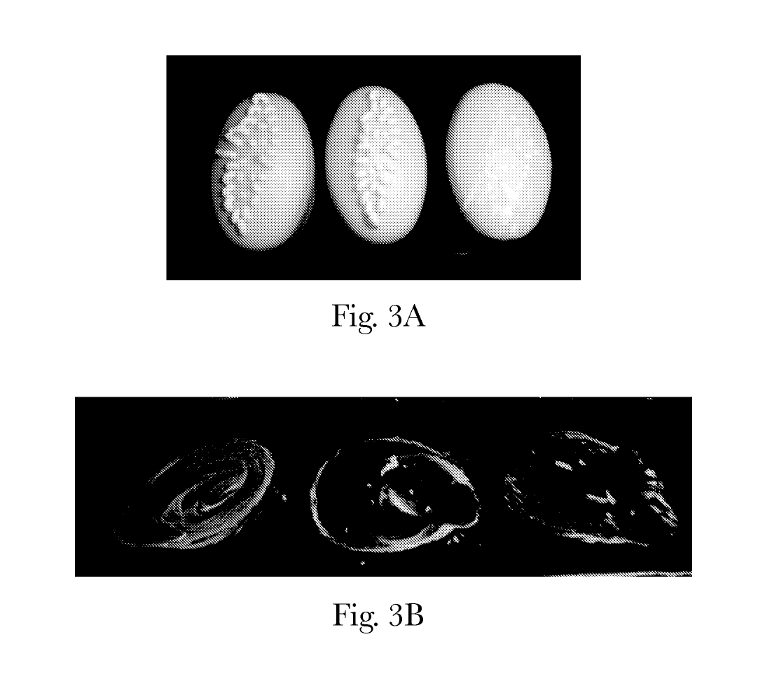

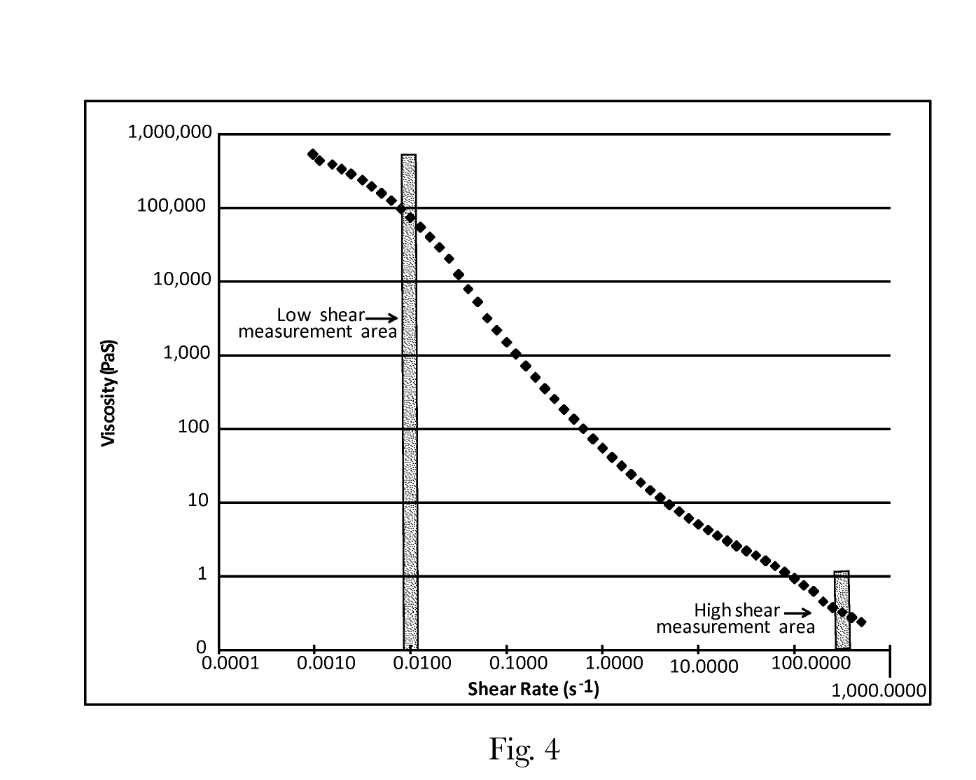

In addition, the type of wax (in combination with the type of carriers and fillers included in the antiperspirant composition) may have an impact on syneresis and/or ease of dispensing of the antiperspirant composition and/or balling of the composition on wet skin/hair. Using only polar waxes provided a composition that can be more susceptible to syneresis at low wax levels, such as 2.5% w/w, in combination with a non-polar only liquid carrier, such as a silicone fluid. An example of what happens at lower wax levels in a polar only wax composition with non-polar liquid carriers can be seen in FIG. 3A, where the polar only wax composition (on the far right) is weeping (syneresis) through the apertures in the dispensing portion of the package. This composition comprised aluminum chlorohydrate (28% w/w), hydrophilic tapioca starch (19% w/w), 50 centistoke dimethicone (5% w/w), petrolatum (3% w/w), betacyclodextrins (3% w/w), stearyl alcohol (2.5% w/w), cyclopentasiloxane (38.75% w/w) and fragrance (0.75% w/w).

It is believed that using only non-polar waxes in combination with a non-polar carrier may avoid syneresis, as shown by the composition shown in the middle of FIG. 3A. This composition comprised aluminum chlorohydrate (28% w/w), hydrophilic tapioca starch (19% w/w), 50 centistoke dimethicone (5% w/w), petrolatum (3% w/w), betacyclodextrins (3% w/w), hydrogenated high euricic acid rapeseed oil (2% w/w), Syncrowax HGLC (0.5% w/w), cyclopentasiloxane (38.75% w/w) and fragrance (0.75% w/w).

However, utilizing only non-polar waxes may have an additional downside. Namely, the antiperspirant composition may “ball up” or become gritty upon application to wet skin or moist axillia hair when used with a hydrophilic filler added as a raw material. The impact of applying an antiperspirant composition to wet skin is shown in FIG. 3B, wherein 0.2 g of a composition was spread on a piece of naugahyde (an artificial leather material available from Uniroyal Engineered Products LLC), pipeting 10 microliters of water onto the soft solid composition, and mixing the water and soft solid composition. The middle swatch of FIG. 3B (containing the same non-polar wax only wax compositions of FIG. 3A) shows a gritty or balled up appearance (small balls are visible) when subjected to the above-described method. The best composition stability (low/no syneresis) and spreadability/low balling was observed when the composition contained a combination of polar and non-polar waxes, as can be seen in the far left swatches of FIGS. 3A and 3B. This composition comprised aluminum chlorohydrate (28% w/w), hydrophilic tapioca starch (19% w/w), 50 centistoke dimethicone (5% w/w), petrolatum (3% w/w), betacyclodextrins (3% w/w), hydrogenated high euricic acid rapeseed oil (1.2% w/w), Syncrowax HGLC (0.3% w/w), stearyl alcohol (1% w/w), cyclopentasiloxane (38.75% w/w) and fragrance (0.75% w/w). Without intending to be bound by any theory, it is believed that the polar wax blend interacts with the hydrophilic filler to protect it from the effects of water. Surprisingly, the polar wax only composition, in the presence of syneresis, also showed balling as seen in the far right swatch of FIG. 3B. Without intending to be bound by any theory, it is believed that synersis, in some instances, may negate the positive effect of including a polar wax in an antiperspirant composition comprising a hydrophilic filler. As such an antiperspirant composition may preferably comprise from about 1.5% to about 5%, by weight of the composition, of a non polar wax and from about 0.5% to about 2%, by weight of the composition, of a polar wax, particularly where a hydrophilic filler is also included in the antiperspirant.

Use of only a non-polar wax may be acceptable in instances where the antiperspirant composition comprises fillers consisting essentially of or completely of hydrophophobic or moderately hydrophobic powders added as a raw material.

Waxes may be natural or synthetic materials. Some examples include natural vegetable waxes such as, for example, candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. Natural waxes may include the mineral waxes, such as ceresine and ozocerite for example, or the petrochemical waxes, for example petrolatum, paraffin waxes and microwaxes. Chemically modified waxes may be used, such as, for example, montan ester waxes, sasol waxes and hydrogenated jojoba waxes. Synthetic waxes include, for example, wax-like polyalkylene waxes and polyethylene glycol waxes.

The wax may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids. Wax components such as these include, for example, C16-40 alkyl stearates, C20-40 alkyl stearates (for example Kesterwachs (Registered trademark K82H), C20-40 dialkyl esters of dimer acids, C18-38 alkyl hydroxystearoyl stearates or C20-40 alkyl erucates. Other suitable waxes which may be used include C30-50 alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Silicone waxes may also be used.

Some preferred examples of acceptable non-polar waxes include glyceryl tribehenate, polyethylene, polymethylene (e.g., Accumelt 68 and 78 available from International Group, Inc., USA), C₁₈-C₃₆ triglyceride (e.g., Synchrowax HGL-C available from Croda, Inc., USA), hydrogenated high euricic aid rapeseed oil (hear stearine), ozokerite and combinations thereof. Some preferred examples of acceptable polar waxes include stearyl alcohol, hydrogenated castor oil, myristyl alcohol, cetyl alcohol, and combinations thereof. The wax may comprise a blend of polar and non-polar waxes. For example, a combination of a polar and non-polar waxes may be selected from the list above.

Antiperspirant Actives

The antiperspirant compositions comprise a particulate antiperspirant active that is insoluble in the liquid ingredients of the antiperspirant composition. While it is desirable to include an antiperspirant active, it will be appreciated that the compositions described herein may also be suitable for deodorant compositions, wherein a deodorant active or agent is substituted for the antiperspirant actives described hereafter. Concentrations of particulate antiperspirant actives can range from about 15%, 20% or 25% to about 35% or 30% by weight of the composition, or any combination thereof. Such weight percentages can be calculated by taking the total active raw material level and multiplying it by the anhydrous assay of the active as determined by the USP method for assay determination (e.g., United States Pharmacopeia 37-National Formulary 32) as commonly known in the art. The antiperspirant active as formulated in the composition can be in a form of dispersed particulate solids. These solids may have an average particle size or equivalent diameter of about 100 microns or less, about 20 microns or less, or about 10 microns or less.

The particulate antiperspirant actives can include any compound, composition, or other particulate material having antiperspirant activity. The antiperspirant actives can include astringent metallic salts. For example, the antiperspirant actives can include inorganic and organic salts of aluminum, zirconium and zinc, as well as mixtures thereof. Antiperspirant active examples can include, but are not limited to, aluminum-containing and/or zirconium-containing salts or materials, such as aluminum halides, aluminum chlorohydrate, aluminum hydroxyhalides, zirconyl oxyhalides, zirconyl hydroxyhalides, and mixtures thereof.

Exemplary aluminum salts can include those that conform to a formula:

Al₂(OH)ₐCl_{b}.xH₂O

wherein a is from about 0 to about 5; a sum of a and b is about 6; x is from about 1 to about 8; where a, b, and x can have non-integer values. For example, aluminum chlorohydroxides referred to as “3/4 basic chlorohydroxide,” wherein a is about 4.5; “5/6 basic chlorohydroxide”, wherein a=5; and “2/3 basic chlorohydroxide”, wherein a=4 can be used. Preferred aluminum salts are referred to as “enhanced” or “improved” or “activated” aluminum chlorohydrate, and as such typically have a high concentration of Band III or Peak IV. Characterization of Band III or Peak IV is well known in the art. Processes for preparing aluminum salts are well known with some examples being disclosed in U.S. Pat. No. 3,887,692, Gilman, issued Jun. 3, 1975; U.S. Pat. No. 3,904,741, Jones et al., issued Sep. 9, 1975; U.S. Pat. No. 4,359,456, Gosling et al., issued Nov. 16, 1982; and British Patent Specification 2,048,229, Fitzgerald et al., published Dec. 10, 1980, all of which are incorporated herein by reference. Mixtures of aluminum salts are described in British Patent Specification 1,347,950, Shin et al., published Feb. 27, 1974, which description is also incorporated herein by reference.

Exemplary zirconium salts can include those which conform to a formula:

ZrO(OH)₂₋ₐClₐ.xH₂O

wherein a is from about 0.5 to about 2; x is from about 1 to about 7; where a and x can both have non-integer values. Such zirconium salts are described in Belgian Patent 825,146, issued to Schmitz on Aug. 4, 1975. The antiperspirant compositions can include zirconium salt complexes that additionally contain aluminum and glycine, commonly known as “ZAG complexes”. Such complexes can contain aluminum chlorohydroxide and zirconyl hydroxy chloride conforming to formulas as set forth above. Preferred include zirconium salt complexes that additionally contain aluminum and glycine are referred to as “enhanced” or “improved” or “activated” aluminum chlorohydrate, and as such typically have a high concentration of Peak IV. Characterization of Peak IV is well known in the art. Such ZAG complexes are described in U.S. Pat. No. 4,331,609, issued to Orr on May 25, 1982 and U.S. Pat. No. 4,120,948, issued to Shelton on Oct. 17, 1978.

Fragrance Delivery Materials

The antiperspirant compositions may comprise fragrance delivery materials that are provided in a particulate form which would be considered part of the total solids concentration of the antiperspirant composition. Examples of some suitable materials to form the fragrance delivery material include, but are not limited to, oligosaccharides (e.g., cyclodextrin), starches, polyethylenes, polyamides, polystyrenes, polyisoprenes, polycarbonates, polyesters, polyacrylates, vinyl polymers, silicas, gelatin, and aluminosilicates. Some examples of fragrance delivery materials are described in U.S. Patent Pub. Nos. 2010/0104611; 2010/0104613; 2010/0104612; 2011/0269658; 2011/0269657; 2011/0268802; and U.S. Pat. Nos. 5,861,144; 5,711,941; 8,147,808; and 5,861,144.

As used herein, the term “cyclodextrin” includes any of the known cyclodextrins such as unsubstituted cyclodextrins containing from six to twelve glucose units, especially alpha-cyclodextrin, beta-cyclodextrin, gamma-cyclodextrin and/or their derivatives and/or mixtures thereof. The term “uncomplexed cyclodextrin” as used herein means that the cavities within the cyclodextrin in the composition of the present invention should remain essentially unfilled prior to application to skin in order to allow the cyclodextrin to absorb various odor molecules when the composition is applied to the skin. While it is desirable that the cyclodextrins incorporated in an antiperspirant composition contain a perfume component, it is contemplated that uncomplexed cyclodextrins may be incorporated as part of the total particulate amount in some instances.

Some cyclodextrins suitable for use in the present invention include alpha-cyclodextrin, beta-cyclodextrin, gamma-cyclodextrin, their derivatives, and mixtures thereof. More preferred are beta cyclodextrin, hydroxypropyl alpha-cyclodextrin, hydroxypropyl beta-cyclodextrin, methylated-alpha-cyclodextrin or methylated-beta-cyclodextrin, and mixtures thereof. Some cyclodextrin complexes, particle sizes, and methods of formation useful herein, are disclosed in U.S. Pat. No. 5,429,628.

B. Low/High Shear Rate Viscosities of the Antiperspirant Compositions

The use of higher solids amounts in a soft solid type antiperspirant composition brings its own challenges. For one thing, having solids about 40% or more, versus below 35% in some commercially available antiperspirant compositions, reduces the amount of available liquid emollient in the antiperspirant composition that carries the active and other powders through axillia hair to the skin. This increases the viscosity of a soft solid antiperspirant composition, making it more difficult to dispense and spread across the entire axilla.

The present inventors have surprisingly discovered soft solid type antiperspirant compositions with a high level of solids that have a particular rheological behavior can provide excellent dispensing and application aesthetics and help offset the need for higher levels of structurants, like wax. Particularly, it has been discovered that formulating a composition to have a low shear rate viscosity of about 25,000 Pa·s to about 90,000 Pa·s may provide sufficient structure for a high solids soft solid composition to avoid unintended flow out of an apertured dome while still providing easy dispensing through the apertures as well as easy spreadability across the entire axillia and dry feel on the skin. Extremely low, low shear rate viscosities, like about 15,000 Pa·s and below, may result in a soft solid composition with wetter feel that is more consistent with a roll-on composition. Moreover, these low shear rate viscosity compositions may lack the structure to be stored in and dispensed from typical soft solids packaging and may (depending on aperture size) run out of the package when tilted for application or stored on their side.

Thus, it is desirable that the antiperspirant composition have a low shear rate viscosity such that the antiperspirant composition does not leak through a plurality of apertures of a dome when the package is inverted (i.e., the apertures are facing downward) and the antiperspirant composition is capable of being extruded through the plurality of apertures when the elevator is advanced toward the dome. An antiperspirant composition may have a low shear rate viscosity from about 25,000 Pa·s, about 30,000 Pa·s, about 35,000 Pa·s, about 40,000 Pa·s, about 45,000 Pa·s to about 90,000 Pa·s, about 80,000 Pa·s, about 75,000 Pa·s, about 70,000 Pa·s, about 65,000 Pa·s, about 60,000 Pa·s, about 55,000 Pa·s, about 50,000 Pa·s or any combination thereof. In one example, the low shear rate viscosity of the composition is from about 40,000 Pa·s to about 60,000 Pa·s. This low shear rate viscosity range is believed to be a function of the method of solidifying the wax, the quantity and type of the wax and liquid carrier, and the solids concentration.

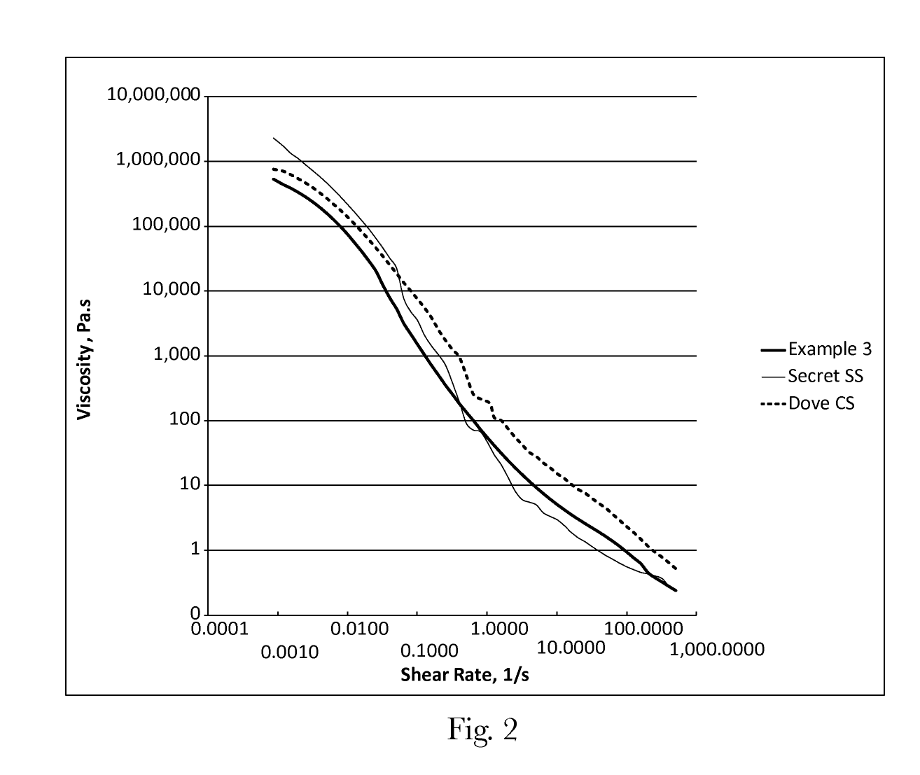

The rheology behavior of some compositions is shown in FIG. 2. As can be seen, some current commercial soft solid compositions have a low shear viscosity above the desired range. For example, Secret Clinical Strength had a low shear rate viscosity of 262,955 Pa·s and Dove Clinical Strength had a low shear rate viscosity of 128,500 Pa·s compared to Example 3 which had a low shear rate viscosity of 74,000 Pa·s. The Dove Clinical Strength composition is believed to have the following ingredients: aluminum zirconium tetrachlorohydrex gly (believed to be 20% anhydrous w/w), cyclopentasiloxane, dimethicone, microcrystalline wax, C18-36 acid triglyceride, silica, fragrance, helianthus annus (sunflower) seed oil, dimethicone crosspolymer, and BHT.

One additional advantage to a low shear rate viscosity of 25,000 Pa·s to about 90,000 Pa·s and a total solids level between about 40% and about 60% is that a dispensing package can be less complicated. Current soft solid compositions may often be delivered through a package where an elevator, on which the composition sits, is ratcheted upward through the twisting of a knob to push composition through apertures in an applicator. In some current soft solid packages, after the composition has been dispensed, the pressure maintained in the package that was created when pushing the composition upward needs to be relieved or the composition will become unstable and syneresis. For example, this problem is addressed by the packaging disclosed in commonly assigned U.S. Pat. No. 5,000,356. The present inventors have also surprisingly discovered that compositions having a sufficient low shear viscosity of 25,000 Pa·s to about 90,000 Pa·s and a total solids level between 40% and 60% do not require the pressure created during dispensing to be relieved through a package feature, like suck back. Thus, the antiperspirant compositions described herein may be stored in packages that are devoid of a mechanism to axially retract the elevator in response to predetermined forward axial movement of the elevator.

Another additional advantage to a low shear rate viscosity of 25,000 Pa·s to about 90,000 Pa·s is that the force required to push the soft solid composition through the apertures in the applicator may be reduced. Pushing the antiperspirant composition through the apertures in the applicator creates a back force on the elevator which increases the torque required to twist the knob that ratchets the elevator upward. This force has been managed in previous soft solid compositions by increasing the total open area of the apertures in the dome of the applicator. Previous soft solid compositions may often needed open surface area of 12% or more to prevent or minimize syneresis. Compositions with a low shear viscosity of 25,000 Pa·s to 90,000 Pa·s may be delivered through a perforated cap or dome with as low as 2% open area. This may be particularly beneficial for achieving lower dosing of the antiperspirant composition, which is discussed more hereafter.

High shear rate viscosity characteristics may also be important to antiperspirant composition acceptance by impacting how the composition spreads through axilla hair and onto skin. A high shear rate is created by rubbing the applicator on the axillia skin with the shear rate being determined by speed of rubbing and the closed area of the applicator. It is believed that, for many consumers, the shear rate is between 100 and 10,000 s⁻¹ during application. High shear rate viscosity is generally affected by the amount and types of solids, with higher solids creating higher viscosity. Also rod shaped are believed to be less desirable as this shape may interact more with each other and the antiperspirant active particulates. As such, starches are believed to be more preferred than cellulose materials.

It is believed that lower high shear rate viscosities, like less than about 1 Pa·s, are better for spreading through hair. The high shear rate viscosity could further be less than 0.5 Pa·s. As can be seen in FIG. 2, two commercial soft solid compositions with 35% or less of solids approach the desired high shear rate viscosity of 1 Pa·s or less, however, they do not provide a desired high and low rate shear viscosity combination. It should be noted that the ability to use packages with lower open areas, or conversely more closed area, increase the shear created by the package during application and further improves spreading of the antiperspirant composition.

C. Carriers

The antiperspirant compositions comprise one or more carriers for suspending, carrying or transferring the antiperspirant active and fillers through hair to the skin. Suitable carries may include liquids and semi-solid materials. The most preferred carriers are emollients.

While increasing the solids concentration may be beneficial for improving the feel of a soft solid antiperspirant composition, it is not without some potential tradeoffs. For example, increasing the solids concentration may lead to increased flaking of the antiperspirant composition from the skin. It is believed this tradeoff may be reduced by including a sufficient amount of one or more non-volatile carriers, preferably non-volatile liquid(s) or emollients, and more preferably a non-volatile silicone liquid and/or mineral oil or mineral oil jelly (e.g., petrolatum) or other non-volatile emollient, in the antiperspirant composition. Other liquids and/or emollients may also be included. For example, in some instances it may be desirable to include a combination of non-volatile and volatile emollients, wherein the combination aids delivery of the antiperspirant active and the fillers, and the volatile emollient evaporates thereby enhancing the dry feel of the antiperspirant composition while the remaining non-volatile emollient aids retentions of the high solids concentration on the skin.

Suitable liquids and/or emollients can include, but are not limited to, organic, silicone-containing or fluorine-containing, volatile or non-volatile, polar or non-polar liquids and/or emollients. Total concentration of the carriers in antiperspirant compositions can typically range from about 35%, 40, 45%, or 50% to about 60% or 55%, by weight of an antiperspirant composition.

In one example, an antiperspirant composition can include one or more silicone liquids. The total concentration of the silicone liquids may range from about 35% to about 60% of the one or more silicone liquids, by weight of the composition. Preferably, the one or more silicone liquids have a concentration from about 35% to about 45% by weight of the composition, optionally in combination with about 1% to about 5% by weight of the composition of a mineral oil or mineral oil jelly (e.g., petrolatum). Suitable silicone liquids include volatile or non-volatile silicones.

Non-limiting examples of suitable silicone liquids for use herein can include volatile silicones described in Todd et al., “Volatile Silicone Fluids for Cosmetics”, Cosmetics and Toiletries, 91:27-32 (1976). Suitable amongst these volatile silicones can include cyclic silicones having from about 3 or from about 4 to about 7 or to about 6, silicon atoms. Suitable silicon carriers can include those which can conform to a formula:

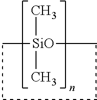

wherein n can be from about 3, from about 4 or from about 5 to about 7 or to about 6. Such volatile cyclic silicones can have a viscosity value of about 10 centistokes or less. Other suitable silicone emollients for use herein can include volatile and nonvolatile linear silicones which conform to a formula:

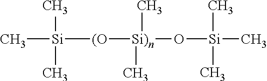

wherein n is greater than or equal to 0. Such volatile linear silicone materials can have viscosity values of about 5 centistokes or less at 25° C. Non-volatile linear silicone materials can have viscosity values of about 5 centistokes or greater at 25° C.

Suitable volatile silicones for use herein can include, but are not limited to, hexamethyldisiloxane; Silicone Fluids SF-1202 and SF-1173 (commercially available from G.E. Silicones); Dow Corning 244, Dow Corning 245, Dow Corning 246, Dow Corning 344, and Dow Corning 345, (commercially available from Dow Corning Corp.); Silicone Fluids SWS-03314, SWS-03400, F-222, F-223, F-250, and F-251 (commercially available from SWS Silicones Corp.); Volatile Silicones 7158, 7207, 7349 (available from Union Carbide); Masil SF-V™ (available from Mazer); and mixtures thereof. The volatile silicone liquids may have a concentration from about 20% to about 40% or about 25% to about 35% by weight of the antiperspirant composition.

Suitable non-volatile linear silicones for use herein can include, but are not limited to, Rhodorsil Oils 70047 available from Rhone-Poulenc; Masil SF Fluid available from Mazer; Dow Coming 200 and Dow Coming 225 (available from Dow Corning Corp.); Silicone Fluid SF-96 (available from G.E. Silicones); Velvasil™ and Viscasil™ (available from General Electric Co.); Silicone L-45, Silicone L-530, and Silicone L-531 (available from Union Carbide); and Siloxane F-221 and Silicone Fluid SWS-101 (available from SWS Silicones). The non-volatile silicone liquid may have a concentration from about 5% to about 20%, or about 5% to about 15% or about 5% to about 10% by weight of the antiperspirant composition.

Other suitable non-volatile silicone liquids for use in antiperspirant soft solid compositions can include, but are not limited to, non-volatile silicone liquids such as polyalkylarylsiloxanes, polyestersiloxanes, polyethersiloxane copolymers, polyfluorosiloxanes, polyaminosiloxanes, and combinations thereof. Such non-volatile silicone liquids can have viscosity values of less than about 100,000 centistokes, less than about 500 centistokes, or from about 1 centistokes to about 200 centistokes or to about 50 centistokes, as measured under ambient conditions. The viscosity of the silicone liquids should be selected to achieve the low and high shear rate viscosities described herein. In many instances, it is desirable for the viscosity of the silicone liquid to be between about 50 centistokes and 200 centistokes to achieve the shear rate viscosities described herein. Small amounts (e.g., less than 1% by weight of the antiperspirant composition) of high viscosity silicone gums having viscosities greater than 100,000 centistokes may also be incorporated in the antiperspirant compositions as a carrier.

Other suitable carriers for use in antiperspirant soft solid compositions can include, but are not limited to, organic emollients such as mineral oil, petrolatum, isohexadecane, isododecane, various other hydrocarbon oils, and mixtures thereof. In one embodiment, mineral oil and branched chain hydrocarbons having from about 4 or from about 6 carbon atoms to about 30 or to about 20 carbon atoms can be suitable emollients. Specific non-limiting examples of suitable branched chain hydrocarbon oils can include isoparaffins available from Exxon Chemical Company as Isopar C™ (C₇-C₈ Isoparaffin), Isopar E™ (C₈-C₉ Isoparaffin), Isopar G™ (C₁₀-C₁₁ Isoparaffin), Isopar H™ (C₁₁-C₁₂ Isoparaffin), Isopar L™ (C11-C13 Isoparaffin), Isopar M™ (C₁₃-C₁₄ Isoparaffin), and combinations thereof. Other non-limiting examples of suitable branched chain hydrocarbons can include Permethyl™ 99A (isododecane), Permethyl™ 102A (isoeicosane), Permethyl™ 101A (isohexadecane), and combinations thereof. The Permethyl™ series are available from Preperse, Inc., South Plainfield, N.J., U.S.A. Other non-limiting examples of suitable branched chain hydrocarbons can include petroleum distillates such as those available from Phillips Chemical as Soltrol™ 130, Soltrol™ 170, and those available from Shell as Shell Sol 70, -71, and -2033, and mixtures thereof.

Suitable organic emollients can include a Norpar™ series of paraffins available from Exxon Chemical Company as Norpar™ 12, -13, and -15; octyldodecanol; butyl stearate; diisopropyl adipate; dodecane; octane; decane; C₁-C₁₅ alkanes/cycloalkanes available from Exxon as Exxsol™ D80; C₁₂-C₁₅ alkyl benzoates available as Finsolv-TN™ from Finetex; and mixtures thereof. Other suitable emollients can include benzoate co-solvents, cinnamate esters, secondary alcohols, benzyl acetate, phenyl alkane, and combinations thereof.

D. Optional Ingredients

In some instances, it may be desirable to include a low concentration of a hydrophilic water insoluble polar oil as a processing aid during the making process. However, these materials may also tend to interfere with active release and may negatively impact efficacy due, at least in part, to their affinity for the antiperspirant active. Thus, the amount of water insoluble hydrophilic oils are preferably minimized to about 5% or less, or about 3% or less, or about 2% or less by weight of the antiperspirant composition. Some water insoluble polar hydrophilic oils include hexyldecanol, PPG-14 butyl ether, octyl decanol and lauryl alcohol. Further, an antiperspirant composition may be substantially free of or free of hydrophilic water insoluble polar oils.

Antiperspirant active efficacy can be measured by the red dot method, set out below, where higher numbers show better efficacy. The negative impact of water insoluble hydrophilic polar oils on efficacy may be demonstrated by looking at the red dot measurements for Comparative Examples 10 and 11 versus inventive Example 9 as they have the same antiperspirant active and concentration. The red dot efficacy results show Comparative Examples 10 and 11 have much lower values. These lower numbers are, without being limited by theory, believed to indicate lower antiperspirant efficacy due to binding of the antiperspirant active by the water insoluble hydrophilic polar oil thereby altering the active's interaction with water.

Antiperspirant soft solid compositions can alternatively or additionally include a deodorant active. Suitable deodorant actives can be selected from the group consisting of antimicrobial agents (e.g., bacteriocides, fungicides), malodor-absorbing material, and combinations thereof. For example, antimicrobial agents can comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, for example, zinc salts, and acids thereof, heavy metal salts of pyrithione, especially zinc pyrithione, zinc phenolsulfate, farnesol, and combinations thereof. Some of these deodorant actives can be solids. When they are in the solid form, then they are counted as part of the total solids.

As discussed above, there are several benefits to the present antiperspirant compositions. For example, an antiperspirant composition comprising from 40% to about 60%, by weight of the composition, of solids comprising: a) an antiperspirant active, b) a wax, and c) a filler; and from about 35% to about 60%, by weight of the composition, of total, non-water liquids, wherein the composition has a low shear viscosity of about 25,000 Pa·s to about 90,000 Pa·s, and a tack value of less than about 60 gF may be beneficial for decreasing tackiness.

As another example, an antiperspirant composition comprising from about 40% to about 60%, by weight of the composition, of solids comprising: a) an antiperspirant active, b) a wax, and c) a filler; and from about 35% to about 60%, by weight of the composition, of total non-water liquids, wherein the composition has a low shear viscosity of about 25,000 Pa·s to about 90,000 Pa·s may improve the dispensability of an anhydrous antiperspirant composition from a package with about 85% to about 98% closed area on a dispensing portion of the package.

As yet another example, an antiperspirant composition comprising from about 40% to about 60%, by weight of the composition, of solids comprising: a) an antiperspirant active, b) a wax comprising from about 1.5% to about 5% of a non-polar wax and about 0.5% to about 2% of a polar wax, and c) a filler; and from about 35% to about 60%, by weight of the composition, of non-water liquids may improve the spreadability of an anhydrous soft solid antiperspirant composition.

An antiperspirant composition comprising from about 40% to about 60%, by weight of the composition, of solids comprising: a) an antiperspirant active, b) a wax, and c) a filler comprising a hydrophilic powder added as a raw material, an hydrophobic or moderately hydrophobic powder added as a raw material, a hydrophilic powder modified to be moderately hydrophobic during the making process, or a combination thereof; and from about 35% to about 60%, by weight of the composition, of non-water liquids may improve the spreadability of an anhydrous soft solid antiperspirant composition on wet skin or through hair.

### II. Packages

The antiperspirant compositions are stored within a dispensing package or applicator. The package is preferably configured to dispense a dose of the antiperspirant composition that is less than 0.4 g and more preferably between about 0.1 g and about 0.3 g or between about 0.15 g and about 0.3 g, although higher doses may also be dispensed. It is believed that reducing the dosage of the antiperspirant composition can further improve the dry feel of the composition as well as help reduce the appearance of residue in a high solids antiperspirant composition. The dosing is a function of the total open area of the apertures of the package through which the antiperspirant composition is dispensed, the amount of elevator travel per rotation of the feed screw and the amount of rotation of the feed screw, as discussed more hereafter. It is believed that less aperture open area may have an additional benefit beyond controlling dose. The increased solid surface area of an aperture dome may assist with providing more effective contact area with the antiperspirant composition during the rub-in in the axillia, thereby increasing the amount of shear during rub in and improving the spreading of the antiperspirant composition across the entire axillia.

As dosage is reduced, it is believed there are some consumer perceived tradeoffs that should be managed. For example, too many small apertures may result in short noodle heights (the noodle being the strand of antiperspirant composition extruded thru the aperture) that may result in the perception that an insufficient amount of antiperspirant composition has been dispensed, thereby leading to over dosing as the consumer continues to extrude more antiperspirant composition to compensate for the visual impression created by short noodle heights from a small number of apertures. In addition, apertures that are too small may lead to syneresis and/or the resistance to extruding the antiperspirant composition through the apertures may be too high. Thus, it is believed that, in some embodiments, there may be desirable aperture open areas, elevator travel and/or aperture configurations (e.g., diameter and total number of apertures) that balance these considerations for the antiperspirant compositions described herein.

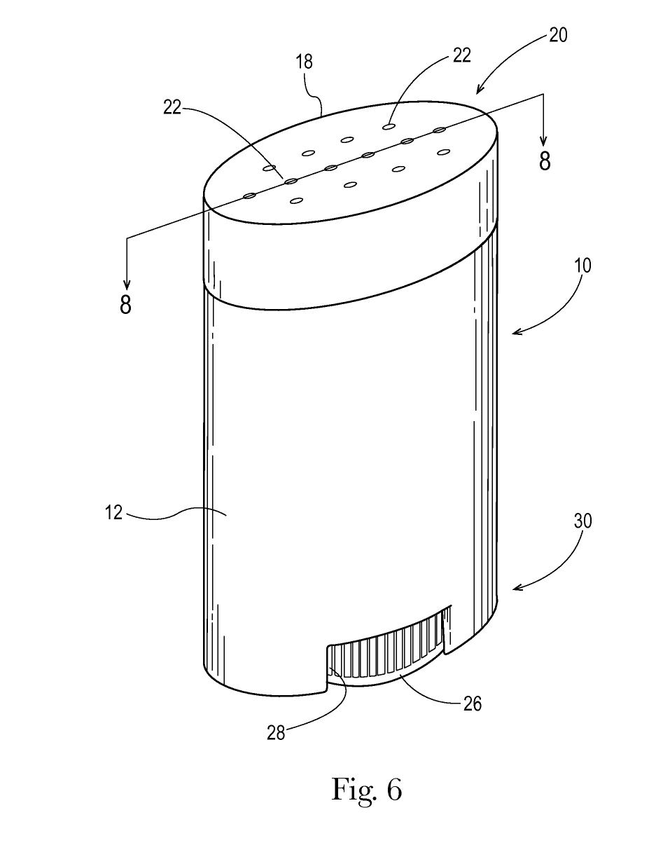

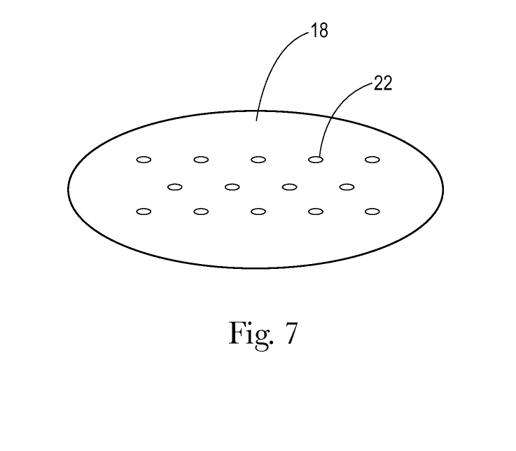

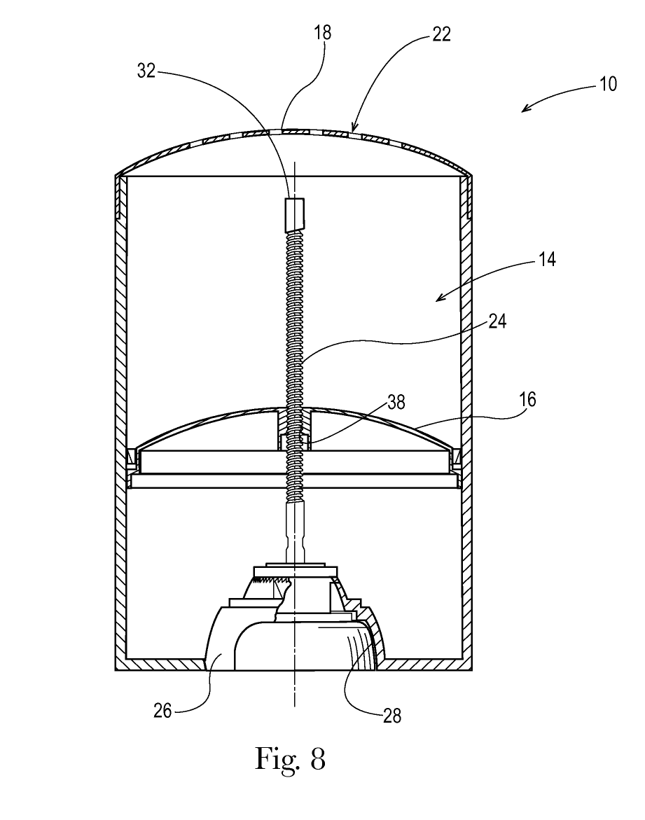

Referring to FIGS. 6 to 8, one example of a dispensing package 10 is shown that is suitable for use with the antiperspirant compositions described herein. The package 10 comprises a container body 12 having an interior chamber 14 which may be of generally uniform or symmetrical cross section and which contains a soft solid type antiperspirant composition. An elevator 16 having a cross section congruent to the interior chamber 14 is mounted for axial movement within the interior chamber 14. A dome 18 is affixed or attached to a first or dispensing end 20 of the container body 12. The dome 18 has a plurality of apertures 22 extending through the thickness of the dome 18. In some embodiments, the surface of the dome 18 may be convexly (or slightly convexly) shaped or flat, although other surface shapes may be provided.

A means for axially advancing the elevator 16 toward the dome 18 is also provided. Such means are well known in the packaging and antiperspirant art and may comprise a feed screw 24 or other similar functioning mechanisms which drive the elevator 16 in an axial direction toward the dome 18. The elevator 16 typically represents the bottom of the dispensing package on or above which the antiperspirant cream composition rests prior to dispensing.

Briefly, the threaded feed screw 24 may be actuated by rotating a hand wheel 26 or other mechanism coupled to the feed screw 24. In use, the hand wheel 26 is rotated, thereby rotating the feed screw 24. Since the elevator 16 is housed within the container body 12, rotation of the feed screw 24 causes the elevator 16 to move up the feed screw 24, thus forcing the antiperspirant composition through the apertures 22 in the dome 18. The hand wheel 26 is held within a recess 28 formed externally of a second end 30 of the container body 12. The recess 28 is formed to house the hand wheel 26 therein, while permitting a user to engage and rotate the hand wheel 26 when the user desires to dispense the antiperspirant composition.

The feed screw 24 has a first end 32 and a second end 34. The second end 34 of the feed screw 24 extends through opening 36 in the second end 34 of the container body 12 and is coupled to the hand wheel 26. In this way, the feed screw 24 is rotated when the hand wheel 26 is rotated by a user. The first end 32 of the feed screw 24 extends within the container body 12 such that the elevator 16 may ride on the feed screw 24 until it reaches a desired position adjacent the first end 32 of the container body 16. The elevator 16 includes a threaded central opening 38 shaped and sized to receive the feed screw 24. Due to the rheological characteristics of soft solid antiperspirant compositions described herein, the package 10 may be devoid of a pressure relief mechanism that automatically relieves residual pressure on the antiperspirant composition, such as by retracting the elevator as described in one or more of U.S. Pat. Nos. 4,356,938 and 5,000,356.

The dome 16 may have from about 4, 10, 20 or 30 to about 60, 40 or 30 apertures 22. In instances where it may be desirable to provide a low dose of the antiperspirant composition to further enhance the light and dry feeling of the composition in use, the dome 16 may have from about 4, 5, or 6 to about 12, 10, 8 or 7 apertures 22. The apertures 22 may be distributed evenly over the dome or not. The dose is a function of the aperture open area, elevator travel per rotation of the feed screw and number of average turns of the dial by a consumer (e.g., typically 2 “clicks” or from about 0.08 to about 0.33 of one full rotation of a feed screw). Where a low dose is desired, the dose is from about 0.1 g, 0.15 g, or 0.2 g to 0.4 g, 0.3 g or 0.25 g with a preferred dose being from about 0.1 g to about 0.3 g. The apertures 22 may be the same size or not. In instances where fewer apertures are desired for low dosing, the total open area of the apertures may be from about 50 mm², 55 mm², 60 mm² to about 100 mm², 90 mm² or 80 mm². The apertures 22 may have a circular or noncircular configuration, preferably a substantially circular (e.g., circular or oval or elliptical) configuration having an average or equivalent circular diameter from about 2 mm to about 6 mm. In instances where fewer apertures 22 are desired for low dosing, the apertures may have an average or equivalent diameter from about 2 mm to about 4 mm. In one preferred configuration, the dome 16 has from about 5 to about 8 apertures, each aperture having an average or equivalent diameter from 2 mm to 4 mm and/or an open area from about 10 mm² to about 14 mm². In some instances, it is believed that the aperture size/configuration may be closer to that used in commercially available gel antiperspirant composition products (e.g., Secret Clear Gel) compared to typical soft solid antiperspirant type products.

The feed screw may be configured to provide an elevator travel, per one full rotation of the feed screw from about 1.5 mm to about 3.75 mm, or 1.5 mm to about 2.75 mm, in combination with the aperture configurations described in order to provide reduced dosing. It is believed these combinations may provide the preferred dosing of less than 0.4 g, more preferably 0.2 g to 0.3 g, in most instances in view of common consumer habits together with a noodle height of the antiperspirant composition per aperture from about 2 mm or 3 mm to about 6 mm, 5 mm or 4 mm. This noodle height provides the consumer with a visual indication that an acceptable amount of the antiperspirant composition has been dispensed and therefore may help avoid over dosing of the composition.

### III. Measurement Methods

Tack Test Method

To determine a tack value for a soft solid type antiperspirant composition, the composition may be analyzed using a texture analyzer, such as Model TA XT plus Texture Analyzer available from Texture Technologies Corp., MA, USA, and having a 30 kg load cell with a 22 mm aluminum probe, a force sensitivity of 1 g, speed range of 0.01 to 40 mm/sec and speed accuracy of better than 0.1%. A fixture for mounting the antiperspirant composition in the load cell may be prepared as follows. First, form a 0.5 inch diameter hole in a piece of acetate sheet (3 inches W×2 inches L). Next, cut a piece of leneta card (e.g., catalog no. N2A-2-Opacity, available from The Leneta Co, N.J., USA) to approximately 3 inches W×2 inches L and place the piece of acetate sheet with 0.5 inch diameter hole over the leneta card. Next, apply the antiperspirant composition directly from the package and spread the composition across the 0.5 diameter hole of the acetate sheet with a spatula if it is necessary (enough to cover inside the hole and 0.5 inches outside the hole). Next, remove the actetate sheet and cut the leneta card down to 2 inches W×1 inch L with the molded antiperspirant composition in the center of the leneta card. Next, attach the leneta card with antiperspirant composition thereon to the base plate of the load cell using double sided adhesive tape. Attach a similarly shaped leneta card (but without the antiperspirant composition) to the probe using double sided adhesive tape. Cycle the load cell for 15 compression cycles using a test distance (i.e., distance between the probe and base plate) of approximately 10 mm. The tack value of the antiperspirant composition is the average value from the testing.

High and Low Shear Rate Viscosity

To determine a viscosity value for a soft solid type antiperspirant composition, the composition may be analyzed using a rheometer, such as Model TA AR2000 available from TA Instruments, New Castle, Del., U.S.A. with data collection using Rheology Advantage Instrument Control Software V5.8.0 and analysis performed using Rheology Advantage Data Analysis Software V5.7.1 (both available from TA Instruments, New Castle, Del., U.S.A.). It will be appreciated that other rheometers and software may be substituted as known in the art. The rheometer is configured in a parallel plate design using a 40 mm serrated upper plate and a serrated lower plate (available as part number 517400.901 from available from TA Instruments, New Castle, Del., U.S.A.) Temperature control is set at 25° C. Analysis of the antiperspirant composition is performed in the “steady state flow” test mode. Rheometer settings are as follows: gap size=1000 microns, shear range 0.001 to 500 1/S, data points collected per decade of shear=10, sample period 10 seconds, percent tolerance=5, consecutive within tolerance=2, and max point time=5 minutes.

Once the rheometer has been appropriately configured and “zeroed”, the antiperspirant composition can be evaluated both before (e.g., low shear rate viscosity) and after shearing through the perforated dome (e.g., high shear rate viscosity) of its package. For compositions being evaluated before shearing through the perforated dome, the dome should be removed before sampling. Approximately 3 grams of composition is sampled and placed in the center of the serrated portion of the lower plate. The upper plate is then lowered to the desired 1000 micron gap, which compresses the composition between the two plates. The entire area between the two plates should be filled. Excess composition (outside the two plates) should be carefully removed with a metal spatula without allowing rotation of the plates or removing composition from between the plates. At this point the sample is ready for analysis.

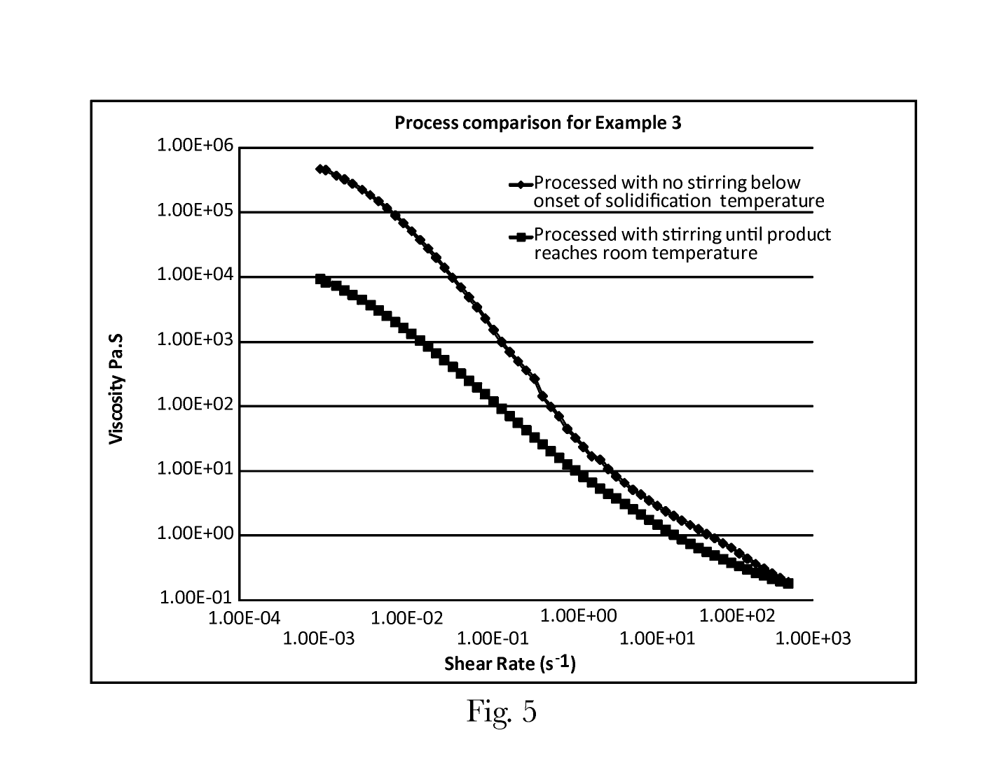

An example data output table is in FIG. 5 and shows a graph of viscosity versus log shear rate for Example 3. Data is plotted in this manner for convenience of viewing the entire data set but low shear rate viscosity will be determined at a shear rate of approximately 0.01 1/S (0.0075 to 0.0125 1/S) and high shear rate viscosity at a shear rate of approximately 315 1/S (310 to 320 1/S). The low shear viscosity measure is believed to be indicative of the viscosity of the soft solid antiperspirant composition in the package during shipping, storage and dispensing through the apertures. The high shear viscosity is believed to be indicative of the viscosity of the soft solid antiperspirant composition during rubbing/spreading across the axillia.

Red Dot Method

The red dot method is one method known in the art for predicting antiperspirant efficacy, and is described further in USPN 2005/0287069. Red dot (or a-values) greater than about 1, 1.25, 1.5, 1.75, or 2 (and less than 5) are believed to be desirable for predicting antiperspirant good efficacy, while values less than 0.5 or even 0 are believed to be less desirable.

A. Preparation of Starting Solutions:

A Phenol Red/Deionized Water solution (Deionized Water, 99.985% per weight; Phenol Red, 0.015% per weight) can be prepared as follows: Add Phenol Red powder to deionized water at room temperature based on the percentages noted. Stir for approximately 2 minutes at approximately 500 rpm, or until the powdered phenol red is completely dissolved into solution, using a magnetic stir bar and stir plate.

A Potassium Hydroxide/Deionized Water solution (Deionized Water, 95% per weight; Potassium Hydroxide (solid), 5% per weight) can be prepared as follows: Add Potassium Hydroxide pellets to deionized water at room temperature based on the noted percentages. Stir for approximately 1 minute at approximately 500 rpm, or until the potassium hydroxide is completely dissolved into solution, using a magnetic stir bar and stir plate.

B. Preparation of pH-Indicator Solution:

The pH-indicator solution can be prepared as follows: Weigh 200 grams of above Phenol Red/Deionized Water solution into a glass beaker. At ambient conditions, stir solution continuously at 100 rpm using a magnetic stir bar and stir plate. Insert calibrated pH probe, such as the Orion 8102BNV from Ross, into the solution. Measure the pH continuously. Adjust pH of Phenol Red/Deionized Water solution to 10.00+/−0.05 by adding 1 ml increments of Potassium Hydroxide/Deionized Water solution to the beaker containing the phenol red/deionized water solution while continually stirring.

C. Preparation of Antiperspirant Sample:

A film of the antiperspirant sample can be prepared using the following procedure. BYTAC TYPE VF-81 chemical resistant Norton FEP film is cut into 3×7 cm rectangles. A circle 2.2 cm in diameter is punched out. The protective back layer of the film is removed and the sticky side of the BYTAC film is adhered to a standard glass microscope slide. Care is taken such that the 2.2 cm circle cut out of the middle of the film is completely on the microscope slide. Antiperspirant is applied on the microscope slide in the center of the circle cut out of the BYTAC film. The antiperspirant sample is thoroughly spread throughout the circle by using a spatula or equivalent in a back and forth motion across the film surrounding the cut out circle. Spreading is continued until a smooth surface of antiperspirant product across the entire cut out circle is achieved. Carefully remove the BYTAC film from the microscope slide leaving behind the smooth, antiperspirant film. The antiperspirant film on the microscope slide is circular with a thickness equal to the thickness of the just removed BYTAC film (about 0.1778 mm).

D. Application of pH Solution:

The antiperspirant film on the microscope slide is dried for about 24 hours at ambient conditions (first drying period). After the first drying period, 20.0 microliters of the phenol red pH-indicator solution are applied to the center of the dried, antiperspirant sample using a standard micropipette such as the 5-50 microliter adjustable Finnpipette from Thermo Lab systems. The sample with the applied 20.0 microliters of phenol red ph indicator solution is left to dry for about 24 hours at ambient conditions (second drying period).

E. Data Collection and Analysis:

After this second 24 hour drying period, the microscope slide with the dried antiperspirant film is placed face up on an approximate 15.24 cm*15.24 cm sample of black felt. A metal ring (1.8 cm diameter*2.5 cm height) is placed on the dried sample eliminating possible contamination of measurements by outside light sources. Care is taken to ensure the dried circle on phenol red ph solution is located in the center of the metal ring. A calibrated Minolta CR-300 series colorimeter, or equivalent, is placed on top of the metal ring. A standard spectral photometric measurement is taken and converted into standard L-a-b scale readings. At least four measurements are taken per sample. The average of the several (at least four) a-value readings is reported.

### IV. Examples

The following examples are given solely for the purpose of illustration and are not to be construed as limitations of the invention as many variations thereof are possible without departing from the spirit and the scope of the invention.

Prophetic Examples 1 and 2 can be made by selecting from within the ranges for each ingredient and then combining the ingredients as follows: The liquids (except fragrance) are added to an appropriate container for heating and their temperature increased to above the melt point of the waxes, to about 85° C. The waxes are added and allowed to completely melt. The solids are then added with agitation that can include milling to remove solid agglomerates. The liquid hot melt may then be cooled to approximately 5-10° C. above the onset of solidification (typically about 60° C. to about 65° C.) and the fragrance is added. The molten liquid antiperspirant composition is then poured into a suitable package at 3° C. to 5° C. degrees above the onset of solidification (typically at 53° C. to 56° C.) and allowed to solidify by cooling to room temperature. The package typically comprises a container body having an elevator and feed screw. After pouring the molten liquid antiperspirant composition into the container body, an apertured dome is attached to the container body. The liquid antiperspirant composition is cooled within the container body to below the solidification onset temperature and to room temperature without stirring, mixing or other significant agitation.

| | Example | Example |
|---|---|---|
| Ingredient | Formulation 1 | Formulation 2 |
| Antiperspirant Active | 15-25% | 15-28% |
| Hydrophilic Powder | 15-25% | 15-45% |
| Nonpolar Wax | 1.5-5% | 1.5-5% |
| Polar Wax | 0.5-2% | 0.5-2% |
| Volatile Emollient | 20-50% | — |
| Nonvolatile Emollient | 5-20% | 20-50% |

Examples 3-9 are antiperspirant soft solid compositions when made by the process described for Example Formulations 1 and 2 above. Comparative Examples 10 and 11 were made by heating the liquids and waxes to 85° C. Adding the solids to the hot melt and cooling with constant agitation (stirring) until the composition reaches room temperature. Additionally Example 3 was remade as Example 3A using the same making methods as Comparative Examples 10 and 11.

| Ingredient | 3 | 3A | 4 | 5 | 6 | 7 | 8 | 9 | 10 | 11 |
|---|---|---|---|---|---|---|---|---|---|---|
| Dimethicone 50 cst | 10.00 | 10.00 | 10.00 | 10.00 | 10.00 | 10.00 | 10.00 | 10.00 | | |
| Petrolatum | 3.00 | 3.00 | 3.00 | 3.00 | | 3.00 | 3.00 | 3.00 | | |
| Cyclopentasiloxane | 32.75 | 32.75 | 32.75 | 32.75 | 38.75 | 32.75 | 32.75 | 37.75 | 29.4 | 27.4 |
| Hear Stearine | 1.20 | 1.20 | 1.20 | 1.20 | 2.00 | 1.20 | 1.20 | 1.20 | | |
| Syncrowax HGL-C | 0.30 | 0.30 | 0.30 | 0.30 | 0.50 | 0.30 | 0.30 | 0.30 | | 3 |
| Stearyl Alcohol | 1.00 | 1.00 | 1.00 | 1.00 | | 1.00 | 1.00 | 1.00 | | |
| Lauryl Alcohol | 1.00 | 1.00 | 1.00 | 1.00 | | 1.00 | 1.00 | 1.00 | | |
| Beta Cyclodextrin | 3.00 | 3.00 | 3.00 | 3.00 | 3.00 | 3.00 | 3.00 | 3.00 | | |
| Tapioca Pure | 22.00 | 22.00 | 19.00 | 33.00 | | | | 22.00 | | |
| Dry Flo TS | | | | | 18.50 | | | | | |
| Aluminum | | | 28.00 | 14.00 | | | 28.00 | 20.00 | 20 | 20 |
| Chlorohydrate | | | | | | | | | | |
| Aluminum | 25.00 | 25.00 | | | 26.5 | 25.00 | | | | |
| Zirconium | | | | | | | | | | |
| Trichlorohydrex | | | | | | | | | | |
| Glycine | | | | | | | | | | |
| Hexadecanol | | | | | | | | | 17 | 17 |
| Hydrogenated | | | | | | | | | 3 | 3 |
| Castor oil | | | | | | | | | | |
| Ceteareth-30 | | | | | | | | | 3 | 3 |
| Peg-20 glycerol | | | | | | | | | 6 | 6 |
| stearate | | | | | | | | | | |
| Modified rice starch | | | | | | | | | 8 | 2 |
| Vitacel CS 20 FC | | | | | | | 19 | | 2 | 2 |
| Silica | | | | | | | | | 1.5 | 1.5 |
| Talc | | | | | | 22.00 | | | 10 | 10 |
| BHT | | | | | | | | | 0.1 | |
| Aluminum Starch | | | | | | | | | | 5 |
| Succinate | | | | | | | | | | |
| Tocopherol Acetate | | | | | | | | | | 0.1 |
| Fragrance | 0.75 | 0.75 | 0.75 | 0.75 | 0.75 | 0.75 | 0.75 | 0.75 | | |
| Total Solid | 52.5 | 52.5 | 52.5 | 52.5 | 50.5 | 52.5 | 52.5 | 52.5 | 47.5 | 47.5 |
| Wax level | 2.5 | 2.5 | 2.5 | 2.5 | 2.5 | 2.5 | 2.5 | 2.5 | 6 | 9 |
| Percent water | 1 | 1 | 1 | 1 | 0 | 1 | 1 | 1 | 17 | 17 |
| Insoluble | | | | | | | | | | |
| Hydrophilic Oil | | | | | | | | | | |
| Low Shear | 74320 | 3392 | 51290 | 39370 | 87540 | 42780 | 237100 | 32410 | 84220 | 161600 |
| Viscosity | | | | | | | | | | |
| High Shear | 0.32 | 0.52 | 1.0 | 1.42 | 0.55 | 0.93 | 1.19 | 0.56 | 0.32 | 0.27 |
| Viscosity | | | | | | | | | | |
| Tack | 56 | | 60.3 | 53.1 | 105.9 | 87.6 | 40.22 | 57.38 | 202.5 | 177.6 |
| Red Dot Test | 2.44 | | 2.06 | 3.6 | 1.98 | 2.21 | 2.79 | 1.98 | −0.09 | −0.05 |

Comparing the compositions of Examples 3 and 3A, which comprise the same ingredients but are processed differently, it can be seen that stirring down to room temperature significantly reduced the viscosity of the composition of Example 3A compared to Example 3 (3,392 Pa·s compared to 74,320 Pa·s). FIG. 5 also shows the impact of process on Example 3A compared to Example 3 across shear rates. In fact, comparative Example 3A might be better described as flowable viscous liquid (as opposed to a soft sold) and would not be suitable for use in packages comprising some apertured domes, as the antiperspirant composition would be capable of leaking through the apertures when the elevator is fully retracted and the package is turned upside down so that the apertures are facing downward. To make this a paste with a higher viscosity, one would need to increase the wax level to above 5% w/w and/or add additional thickener, such as silica, and/or add significant concentrations of water insoluble hydrophilic oil to control for syneresis. Example 3 had an acceptable low shear rate viscosity, red dot value and did not exhibit syneresis upon dispensing.

Comparative Examples 10 and 11, which were made according to the same process as comparative Example 3A, comprised higher wax concentrations and a polar, water insoluble hydrophilic oils (e.g., hexyldecanol). While these compositions had significantly higher low shear rate viscosities than Example 3A, both Examples 10 and 11 suffered from low red dot values which might be indicative of relatively poorer antiperspirant efficacy compared to Example 3A and inventive Example 9, the latter having the same antiperspirant active and concentration but made according to the same process as inventive Example 3. Example 9 had an acceptable low shear rate viscosity, red dot value and did not exhibit syneresis upon dispensing.

Example 6 comprised, inter alia, a moderately hydrophobic starch and no polar wax. This composition had an acceptable low shear viscosity, although the composition was becoming more difficult to dispense, and an acceptable red dot value. No syneresis was observed on dispensing.

Example 7 comprised, inter alia, talc and polar waxes, although it is believed that the polar waxes were not needed in this Example. The composition had acceptable low shear viscosity, red dot value and no syneresis was observed upon dispensing.

Example 8 comprised, inter alia, a cellulose powder and polar waxes. The composition had a very high low shear rate viscosity, most likely due to the cellulose, and an acceptable red dot value.

This application claims the benefit of U.S. Provisional Application Ser. No. 61/729,867, the entirety of which is incorporated by reference herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A packaged antiperspirant product, comprising:
an anhydrous antiperspirant composition, comprising:
from about 40% to about 60%, by weight of the anhydrous antiperspirant composition, of solids comprising:
a) an antiperspirant active,
b) one or more waxes, and
c) one or more fillers having a total concentration from about 15% to about 35% by weight of the anhydrous antiperspirant composition; and
from about 35% to about 60% by weight of the composition, of one or more carriers, wherein the one or more carriers comprises a non-volatile silicone liquid; and
a package comprising a container body having an interior chamber with the anhydrous antiperspirant composition stored therein, a dome closing one end of the container body and comprising a plurality of apertures extending through the thickness of the dome, an elevator disposed within the container body for pushing the anhydrous antiperspirant composition toward the dome and a feed screw threadably engaging the elevator for advancing the elevator toward the dome.

2. The packaged antiperspirant product according to claim 1, wherein the anhydrous antiperspirant composition has a low shear rate viscosity such that the anhydrous antiperspirant composition does not leak through the plurality of apertures when the package is inverted and the anhydrous antiperspirant composition is capable of being extruded through the plurality of apertures when the elevator is advanced toward the dome.

3. The packaged antiperspirant product according to claim 1, wherein the anhydrous antiperspirant composition has a low shear rate viscosity from about 25,000 Pa·s to about 90,000 Pa·s as measured by a rheometer at ambient conditions and wherein the anhydrous antiperspirant composition is expellable through the apertures of the dome without syneresis.

4. The packaged antiperspirant product according to claim 1, wherein the plurality of apertures have a total open area from about 50 mm² to about 100 mm².

5. The packaged antiperspirant product according to claim 4, wherein the each of the plurality of apertures have an open area from about 10 mm² to about 14 mm².

6. The packaged antiperspirant product according to claim 4, wherein the plurality of apertures comprises from about 4 to about 12 apertures and the feed screw provides an elevator travel from about 1.5 mm to about 2.75 mm per full rotation of the feed screw.

7. The packaged antiperspirant product according to claim 1, wherein the anhydrous antiperspirant composition is substantially free of silica having more than 100 meters² of surface area per gram, cellulose powders and a combination of an organo modified clay and a clay activator.

8. The packaged antiperspirant product according to claim 1, wherein the solids consist essentially of the antiperspirant active, the one or more waxes, one or more starches, optionally talc, and optionally a fragrance delivery material.

9. The packaged antiperspirant product according to claim 1, wherein the one or more fillers is selected from the group consisting of tapioca starch, corn starch, oat starch, potato starch, wheat starch, talc, perfume delivery materials and combinations thereof.

10. The packaged antiperspirant product according to claim 1, wherein: i) the one or more fillers comprise a hydrophilic starch powder or a clay other than an organo modified clay and the one or more waxes comprises a polar wax and a non-polar wax, or ii) the one or more fillers comprise a hydrophobic powder and the one or more waxes comprises a non-polar wax.

11. The packaged antiperspirant product according to claim 1, wherein the anhydrous antiperspirant composition comprises from about 2% to about 5% by weight of the one or more waxes.

12. The packaged antiperspirant product according to claim 1, wherein the one or more waxes comprise from about 1.5% to about 3%, by weight of the anhydrous antiperspirant composition, of a non-polar wax, and from 0.5% to 2%, by weight of the anhydrous antiperspirant composition, of a polar wax.

13. The packaged antiperspirant product according to claim 1, wherein the anhydrous antiperspirant composition has a tack value less than 80 gF.

14. The packaged antiperspirant product according to claim 1, wherein the one or more carriers further comprises a volatile silicone liquid having a concentration from about 20% to about 40% by weight of the anhydrous antiperspirant composition and wherein the non-volatile silicone liquid has a concentration from 5% to 20% by weight of the anhydrous antiperspirant composition.

15. The packaged antiperspirant product according to claim 1, wherein the anhydrous antiperspirant composition has a continuous wax structure formed by cooling the antiperspirant composition to below its solidification onset temperature while the antiperspirant composition is stored in the interior chamber of the package.

16. A method for making an anhydrous antiperspirant composition, comprising:
forming a liquid anhydrous antiperspirant composition, comprising:
from 40% to about 60%, by weight of the anhydrous antiperspirant composition, of solids comprising:
a) an antiperspirant active,
b) one or more waxes, and
c) one or more fillers having a concentration from about 15% to about 35% by weight of the anhydrous antiperspirant composition; and
from about 35% to about 60%, by weight of the composition, of one or more carriers; and
pouring the liquid anhydrous antiperspirant composition into a package comprising a container body having an elevator and an interior chamber for receiving the liquid anhydrous antiperspirant composition, wherein the anhydrous antiperspirant composition is poured into the container body at a temperature greater than about 40 C; and
cooling the liquid anhydrous antiperspirant composition stored within the interior chamber of the package to form a solid anhydrous antiperspirant composition.

17. The method according to claim 16, wherein the liquid anhydrous antiperspirant composition is poured into the container body at a temperature from about 40° C. to about 80° C.

18. The method according to claim 16, wherein the liquid anhydrous antiperspirant composition is cooled from a temperature greater than about 40° C. to room temperature without agitation to form a solid.

19. A method for applying an anhydrous antiperspirant composition from a package, comprising:
dispensing an anhydrous antiperspirant composition from a package, the anhydrous antiperspirant comprising:
from 40% to about 60%, by weight of the anhydrous antiperspirant composition, of solids comprising:
a) an antiperspirant active,
b) a hydrophobic powder and a non-polar wax or a hydrophilic powder and a combination of a polar wax and a non-polar wax; and
one or more carriers; and
wherein the package comprises a container body having an interior chamber with the anhydrous antiperspirant composition stored therein, a dome closing one end of the container body and comprising a plurality of apertures extending through the thickness of the dome, an elevator disposed within the container body for pushing the anhydrous antiperspirant composition toward the dome and a feed screw threadably engaging the elevator for advancing the elevator toward the dome; and
rubbing the anhydrous antiperspirant composition into the axillia of a male user.

20. The method according to claim 19, further comprising dispensing the anhydrous antiperspirant composition from the package so as to form noodle heights from the plurality of apertures from 2 mm to about 4 mm.
